(19) United States
(12) Patent Application Publication
(10) Pub. No.: US 2009/0254167 A1
(43) Pub. Date: Oct. 8, 2009

(54) BIFURCATED STENT DELIVERY SYSTEM AND METHOD OF USE

(76) Inventors: Donald R. Ricci, Vancouver (CA); George A. Shukov, Los Altos Hills (CA); Ian M. Penn, Vancouver (CA)

Correspondence Address: KATTEN MUCHIN ROSENMAN LLP;(C/O PATENT ADMINISTRATOR), 2900 K STREET NW, SUITE 200, WASHINGTON, DC, 20007-5118, US

(21) Appl. No.: 12/418,890

(22) Filed: Apr. 6, 2009

Related U.S. Application Data
(63) Continuation of application No. 11/458,819, filed on Jul. 20, 2006, now abandoned.
Application No. 11/458,819 is a continuation of application No. 09/744,950, filed on Jun. 18, 2001, now abandoned.

(30) Foreign Application Priority Data
Jul. 20, 1999 (CA) PCT/CA99/00695

(51) Int. Cl. A61F 2/06

(52) U.S. Cl. 623/1.11

(57) ABSTRACT

An endovascular sleeve which can be utilized to navigate a pair of guidewires to a bifurcated body passageway such that, once in place, the guidewires are substantially untwisted or untangled. This greatly facilitates delivery of the bifurcated stent to the bifurcated artery.

### REFERENCE TO RELATED APPLICATIONS

This application is a continuation of U.S. patent application Ser. No. 11/458,819, filed Jul. 20, 2006, which is a continuation of U.S. patent application Ser. No. 09/744,950, filed Jun. 18, 2001 (now abandoned), which is a 371 of international Appln. No. PCT/CA99/00695, filed Jul. 20, 1999, which claims the benefit of U.S. patent Appln. No. 60/094,950, filed Jul. 31, 1998, the contents of which are incorporated herein by reference.

### TECHNICAL FIELD

In one of its aspects, the present invention relates to an endovascular sleeve for use in delivery of a bifurcated stent. In another of its aspects, the present invention relates to bifurcated stent delivery kit. In yet another of its aspects, the present invention relates to a method for delivery of a bifurcated stent.

### BACKGROUND ART

Stents are generally known. Indeed, the term “stent” has been used interchangeably with terms such as “intraluminal vascular graft” and “expansible prosthesis”. As used throughout this specification, the term “stent” is intended to have a broad meaning and encompasses any expandable prosthetic device for implantation in a body passageway (e.g., a lumen or artery).

In the past ten years, the use of stents has attracted an increasing amount of attention due the potential of these devices to be used, in certain cases, as an alternative to surgery. Generally, a stent is used to obtain and maintain the patency of the body passageway while maintaining the integrity of the passageway. As used in this specification, the term “body passageway” is intended to have a broad meaning and encompasses any duct (e.g., natural or iatrogenic) within the human body and can include a member selected from the group comprising: blood vessels, respiratory ducts, gastrointestinal ducts and tho like.

Stent development has evolved to the point where the vast majority of currently available stents rely on controlled plastic deformation of the entire structure of the stent at the target body passageway so that only sufficient force to maintain the patency of the body passageway is applied during expansion of the stent.

Generally, in many of these systems, a stent, in association with a balloon, is delivered to the target area of the body passageway by a catheter system. Once the stent has been properly located (for example, for intravascular implantation, the target area of the vessel can be filled with a contrast medium to facilitate visualization during fluoroscopy), the balloon is expanded thereby plastically deforming the entire structure of the stent so that the latter is urged in place against the body passageway. As indicated above, the amount of force applied is at least that necessary to expand the stent (i.e., the applied force exceeds the minimum force above which the stent material will undergo plastic deformation) while maintaining the patency of the body passageway. At this point, the balloon is deflated and withdrawn within the catheter, and is subsequently removed.

Ideally, the stent will remain in place and maintain the target area of the body passageway substantially free of blockage (or narrowing). See, for example, any of the following patents:

U.S. Pat. No. 4,733,665 (Palmaz),
U.S. Pat. No. 4,739,762 (Palmaz),
U.S. Pat. No. 4,800,882 (Gianturco),
U.S. Pat. No. 4,907,336 (Gianturco),
U.S. Pat. No. 5,035,706 (Gianturco et al.),
U.S. Pat. No. 5,037,392 (Hillstead),
U.S. Pat. No. 5,041,126 (Gianturco),
U.S. Pat. No. 5,102,417 (Palmaz),
U.S. Pat. No. 5,147,385 (Beck et al.),
U.S. Pat. No. 5,282,824 (Gianturco),
U.S. Pat. No. 5,316,023 (Palmaz et al.),

### Canadian Patent No. 1,239,755 (Wallsten),

### Canadian Patent No. 1,245,527 (Gianturco et al.),

Canadian patent application number 2,171,047 (Penn et al.),
Canadian patent application number 2,175,722 (Penn et al.),
Canadian patent application number 2,185,740 (Penn et al.),
Canadian patent application number 2,192,520 (Penn et al.),
International patent application number PCT/CA97/00151 (Penn et al.), and
International patent application number PCT/CA97/00152 (Penn et al.),
the contents of each of which are hereby incorporated by reference, for a discussion on previous stent designs and deployment systems.

All of the stents described in the above-identified patents share the common design of being mono-tubular and thus, are best suited to be delivered and implanted in-line in the body passageway. These known stents are inappropriate for use in a bifurcated body passageway (e.g., a body passageway comprising a parent passageway that splits into a pair of passageways). Further, these stents are inappropriate for use in a body passageway having side branches since: (i) inaccurate placement of the stent substantially increases the risk to the patient, (ii) the risk of passageway closure in the side branches is increased, and (iii) the side branches will be substantially inaccessible.

Indeed, the Physician Guide published in support of the Palmaz-Schatz stent states on page 32 (the contents of which are hereby incorporated by reference):
“ . . . no attempt should be made following placement of a PALMAZ-SCHATZ stent to access the side branch with a guide wire or a balloon, as such attempts may result in additional damage to the target vessel or the stent. Attempts to treat obstructed side branches within stented segments can result in balloon entrapment, necessitating emergency bypass surgery.”
   Thus, when installed, the Palmaz-Schatz stent admittedly shields side branches emanating from the target area of the body passageway effectively permanently. This can be problematic since the only way to treat blockage or other problems associated with the side branches is to perform the type of surgery which installation of the stent was intended to avoid.

This contraindication for conventional mono-tubular stents is corroborated by a number of investigators. See, for example, the following:

1. Interventional Cardiovascular Medicine: Principles and Practice (1994); Publisher: Churchill Livingstone Inc.; pages 221-223 (Ohman et al.), 487-488 (Labinaz et al.), 667-668 (Bashore et al.) and 897 (Bailey et al.), including references cited therein;
2. Gianturco-RoubinFlex-Stent™ Coronary Stent: Physician's Guide; page 2, Paragraph 3 under WARNINGS;
3. Circulation, Vol. 83, No. 1, January 1991 (Schatz et al.); entitled “Clinical Experience With the Palmaz-Schatz Coronary Stent”; pages 148-161 at page 149; and
4. American Heart Journal, Vol. 127, No. 2, February 1994 (Eeckhout et al.); entitled “Complications and follow-up after intracoronarystenting: Critical analysis of a 6-year single-center experience”; pages 262-272 at page 263, the contents of each of which are hereby incorporated by reference.

Further, some investigators have attempted to install individual stents in each branch of the bifurcated body passageway. However, this approach is fraught with at least two significant problems. First, implantation of three individual stents is technically challenging and, together with the expansive forces generated upon implantation, results in subjecting the central walls of the bifurcated body passageway to undue stress and trauma which may lead to postprocedural complications. Second, since the central walls (i.e., in the crotch area) of the bifurcated body passageway are not supported by the individual stents, this area of the passageway is left substantially unprotected and susceptible to blockage.

One particular problem area with bifurcated body passageways is the occurrence of bifurcation lesions within the coronary circulation. Generally, these legions may be classified as follows:

| Type | Characteristic |
|---|---|
| A | Prebranch stenosis not involving the ostium of the side branch; |
| B | Postbranch stenosis of the parent vessel not involving the origin of |
| | the side branch; |
| C | Stenosis encompassing the side branch but not involving the |
| | ostium; |
| D | Stenosis involving the parent vessel and ostium of the side branch; |
| E | Stenosis involving the ostium of the side branch only; and |
| F | Stenosis discretely involving the parent vessel and ostium of the |
| | side branch. |

See the Atlas of Interventional Cardiology (Popma et al.), 1994, pages 77-79, the contents of which are hereby incorporated by reference. The presence of bifurcation lesions is predictive of increased procedural complications including acute vessel closure.

U.S. Pat. No. 4,994,071 (MacGregor), the contents of which are hereby incorporated by reference, discloses a bifurcating stent apparatus. The particular design incorporates a series of generally parallel oriented loops interconnected by a sequence of “half-birch” connections. The lattice structure of the illustrated stent is constructed of wire. The use of such wire is important to obtain the loop structure of the illustrated design. U.S. Pat. Nos. 3,993,078 (Bergentz et al.) and 5,342,387 (Summers), the contents of each of which are hereby incorporated by reference, also disclose and illustrate a bifurcated stent design constructed of wire.

In published Canadian patent application number 2,134,997 (Penn et al.) and published International patent application PCT/CA97/00294 (Penn et al.), the contents of each of which are hereby incorporated by reference, we describe various novel bifurcated stents.

Thus, while bifurcated stents are generally known, the base of knowledge relating thereto is significantly less than that relating to monotubular stents. Not surprisingly there is a similar imbalance of knowledge relating to the delivery systems for such stents. Specifically, there is vast knowledge relating delivery systems for monotubular stents compared to the knowledge that exists for bifurcated stent delivery systems.

In the delivery of any stent (monotubular or bifurcated) it is reasonably well accepted that the stent is mounted on a catheter which is navigated over a guidewire previously inserted through a guide catheter to the target location. Thus, when the object is to deliver a bifurcated stent, it is envisaged that a pair of guidewires would be used—i.e., one for each of the two passageways that branch off the primary passageway. As such, it is important that, in the primary passage, the guidewires do not become entangled, either in the guide catheter or the body passageway, as this will prevent navigation of the catheter to the target location. In addition, the limited size of the guide catheter determines the bulkiness of the bifurcated stent delivery system. The practical result of this is that the current approach of delivering bifurcated stents is bulky, cumbersome, and technically challenging. To date, the present inventors are unaware of a solution to the problems of conventional bifurcated stent delivery.

Accordingly, it would be desirable to have a system which could be used to navigate a pair of guidewires in a substantially untangled manner to facilitate delivery of the bifurcated stent. It would be further advantageous is such a system were relatively miniaturized compared to conventional bifurcated stent delivery systems.

### SUMMARY OF THE INVENTION

It is an object of the present invention to provide a novel bifurcated stent delivery system which obviates or mitigates at least one of the above-mentioned disadvantages of the prior art.

Thus, in one of its aspects, the present invention provides an endovascular sleeve for delivering a pair of guidewires to a bifurcated body passageway, the sleeve comprising a first tubular passageway and a second tubular passageway fixed with respect to one another, the first tubular passageway comprising a first distal end and a first proximal end, the second tubular passageway comprising a second distal end and a second proximal end, the first distal end being longer than the second distal end to define a junction which abuts against a crotch in the bifurcated body passageway.

A bifurcated stent delivery kit for delivery of a bifurcated stent to a bifurcated body passageway, the kit comprising: a catheter; a pair of guidewires; and an endovascular sleeve for delivering the guidewires to a bifurcated body passageway, the sleeve comprising a first tubular passageway and a second tubular passageway fixed with respect to one another, the first tubular passageway comprising a first distal end and a first proximal end, the second tubular passageway comprising a second distal end and a second proximal end, the first distal end being longer than the second distal end to define a junction which abuts against a crotch in the bifurcated body passageway.

In yet another of its aspects, the present invention provides method for delivery of a bifurcated stent to a target bifurcated body passageway having a proximal body passageway, a first distal body passageway and a second distal body passageway using an endovascular sleeve comprising a first tubular passageway and a second tubular passageway fixed with respect to one another, the first tubular passageway comprising a first distal end and a first proximal end, the second tubular passageway comprising a second distal end and a second proximal end, the first distal end being longer than the second distal end to define a junction which abuts against a crotch in the bifurcated body passageway, the method comprising the steps of: (i) navigating a first guidewire through the primary proximal body passageway and into the first distal body passageway; (ii) feeding the first tubular passageway of the endovascular sleeve over the first guidewire; (iii) navigating the endovascular sleeve through the primary proximal body passageway until the first distal end is disposed in the first distal body passageway and the junction abuts a crotch in the bifurcated body passageway; (iv) navigating a second guidewire through the second tubular passageway and into the second distal body passageway; (v) withdrawing the endovascular sleeve from the body passageway; (vi) guiding a catheter over the first guidewire and the second guidewire, the catheter having a bifurcated stent disposed thereon; (vii) navigating the bifurcated stent to the target bifurcated body passageway; and (viii) expanding the bifurcated stent.

Thus, the present inventors have developed an endovascular sleeve which can be utilized to navigate a pair of guidewires to a bifurcated body passageway such that, once in place, the guidewires are substantially untwisted or untangle. This greatly facilitates delivery of the bifurcated stent to the bifurcated artery.

### BRIEF DESCRIPTION OF THE DRAWINGS

Embodiments of the present invention will be described with reference to the accompanying drawings wherein like numerals designate like parts and in which:

FIG. 1 illustrates a side elevation of a first embodiment of the present endovascular sleeve;

FIG. 2 illustrates a side elevation of a second embodiment of the present endovascular sleeve;

FIGS. 3-7 illustrate enlarged views of how the present endovascular sleeve may be used to deliver a pair of guidewires;

FIGS. 8-12 illustrate perspective views of how the present endovascular sleeve may be used to deliver a pair of guidewires;

FIGS. 13-15 illustrate enlarged view of how a bifurcated stent may be delivered once the pair of guidewires are in place; and

FIG. 16 illustrates an enlarged view of the implanted bifurcated stent delivered in FIGS. 13-15.

### BEST MODE FOR CARRYING OUT THE INVENTION

With reference to FIG. 1, there is shown an endovascular sleeve 10. Endovascular sleeve 10 comprises a first tubular passageway 20 having a first distal end 22 and first proximal end 24. Endovascular sleeve 10 further comprises a second tubular passageway 30 having a second distal end 32 and second proximal end 34. First tubular passageway 20 and second tubular passageway 30 are joined and fixed with respect to one another along a seam 40.

As illustrated, first distal end 22 extends beyond second distal end 32. This offset between first distal end 22 and second distal end 32 defines a junction 45 Preferably, first distal end 22 extends beyond second distal end 32 by a margin of at least about 0.3 cm, more preferably by a margin in the range of from about 0.3 cm to about 3 cm, most preferably by a margin in the range of from about 0.5 cm to about 2 cm. Further, first proximal end 24 is significantly offset with respect to second proximal end 34. As will be developed below, this offset renders endovascular sleeve 10 as an “over-the-wire/monorail” delivery system. As shown, each of first distal end 22 and second distal end 32 are chamfered or beveled.

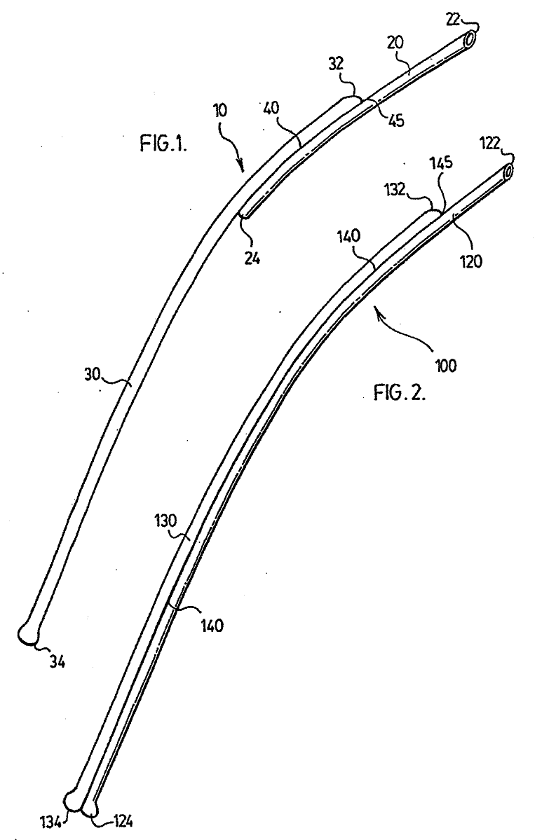

With reference to FIG. 2, there is shown an endovascular sleeve 100. Endovascular sleeve 100 comprises a first tubular passageway 120 having a first distal end 122 and first proximal end 124. Endovascular sleeve 100 further comprises a second tubular passageway 130 having a second distal end 132 and second proximal end 134. First tubular passageway 120 and second tubular passageway 130 are joined and fixed with respect to one another along a seam 140. As illustrated, first distal end 122 extends beyond second distal end 132. This offset between first distal end 122 and second distal end 132 defines a junction 145. Preferably, first distal end 122 extends beyond second distal end 132 by a margin of at least about 0.3 cm, more preferably by a margin in the range of from about 0.3 cm to about 3 cm, most preferably by a margin in the range of from about 0.5 cm to about 2 cm. Further, unlike in the “over-the-wire/monorail” delivery system illustrated in FIG. 1, first proximal end 124 is substantially even with respect to second proximal end 134. This relatively even disposition of first proximal end 124 and second proximal end 134 renders endovascular sleeve 100 as a “double over-the-wire” delivery system. As shown, each of first distal end 122 and second distal end 132 are chamfered or beveled.

The material used to constructed endovascular sleeve 10 is not particularly restricted provided of course that it: (i) sufficient integrity to by navigated through tortuous body passageways, and (ii) is non-toxic to the subject in which endovascular sleeve 10 is being navigated. Non-limiting examples of suitable materials include bioplastic polymers, a flexible metal tube, and the like.

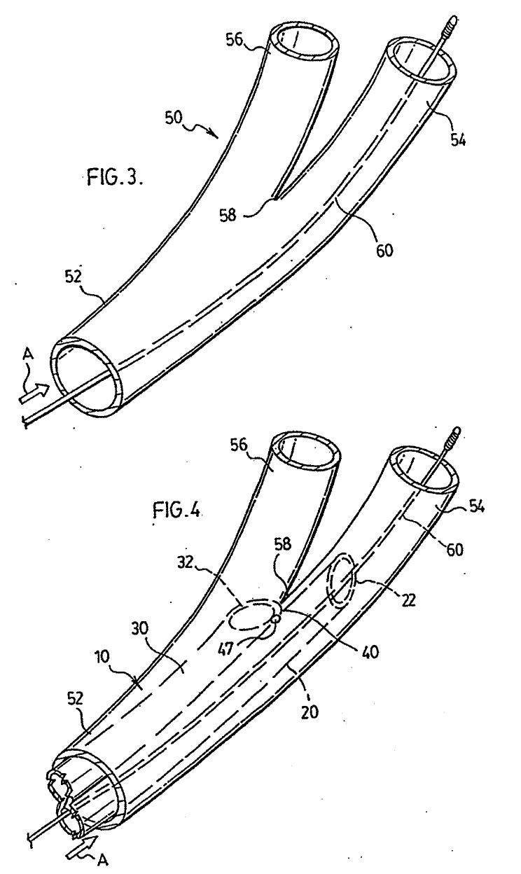

With reference to FIGS. 3-7, the use of endovascular sleeve 10 used to deliver a pair of guidewires will be discussed.

As shown, a bifurcated body passageway 50 comprises a proximal passageway 52 and a pair of distal passageways 54, 56. The junction of distal passageways 54, 56 defines a crotch 58. For clarity, the stenosis of bifurcated body passageway 50 is not illustrated.

With reference to FIG. 3, a first guidewire 60 is navigated through proximal passageway 52 and into distal passageway 54 in the direction of arrow A.

With reference to FIG. 4, first tubular passageway 20 is fed over guidewire 60 in the direction of arrow A and navigated until it enters distal passageway 54 and junction 40 of endovascular sleeve 10 abuts crotch 58 of bifurcated body passageway 50. In the illustrated embodiment, endovascular sleeve 10 is provided with a radioopaque marker (e.g., made of gold and the like) near or at junction 40 so that the position of junction 40 relative to crotch 58 can be monitored using conventional image radiography techniques. Once endovascular sleeve 10 is positioned in this fashion, second distal end 32 of second tubular passageway 30 opens into distal passageway 56.

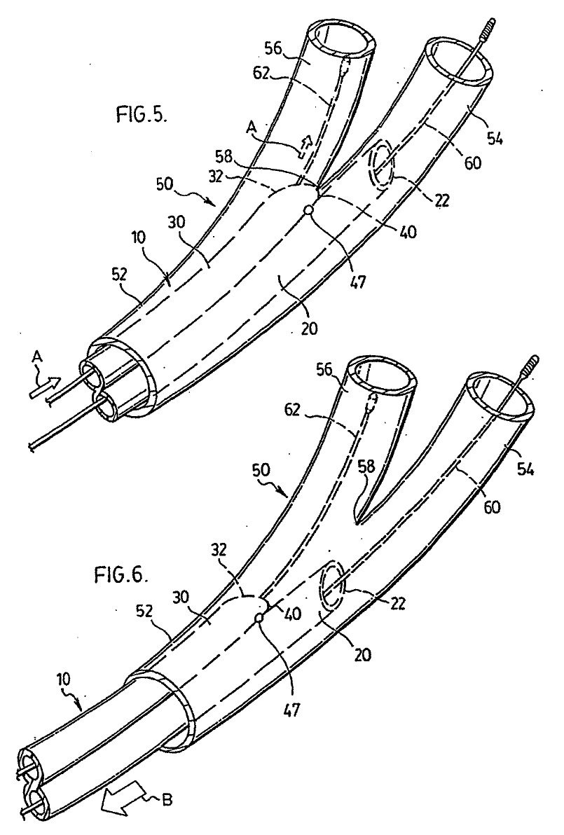

With reference to FIG. 5, once endovascular sleeve 10 is in place (i.e., as shown in FIG. 4), a second guidewire 62 is fed through second tubular passageway 30 into distal passageway 56 in the direction of arrow A.

With reference to FIG. 6, once guidewires 60, 62 are positioned correctly, endovascular sleeve 10 is withdrawn from bifurcated body passageway 50 in the direction of arrow B. As will be apparent to those of skill in the art, care should be taken to avoid twisting of endovascular sleeve 10 since this could result in conveyance of the twist to guidewires 60, 62.

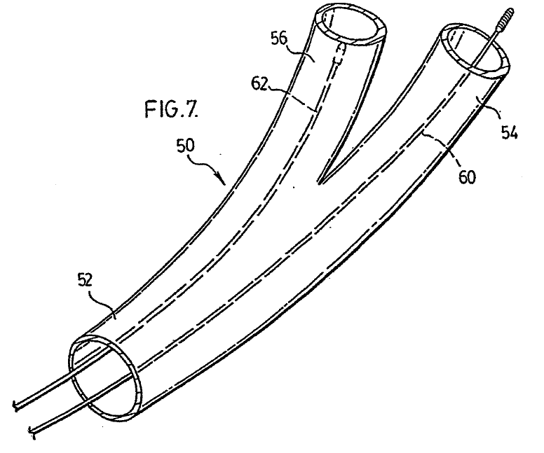

With reference to FIG. 7, once endovascular sleeve 10 is completely withdrawn from bifurcated body passageway 50, guidewires 60, 62 remain with the distal ends thereof in distal passageways 54, 56, respectively.

With reference to FIGS. 8-12, there are illustrated perspective views of the use of endovascular sleeve 10 to deliver a pair of guidewires as described hereinabove with respect to FIGS. 3-7.

As illustrated, endovascular sleeve 10 is introduced to a subject 70 via a suitable incision near the groin of subject 70. Generally speaking, the concordance of the perspectives view illustrated in FIGS. 8-12 to the enlarged view illustrated in FIGS. 3-7 is as follows:

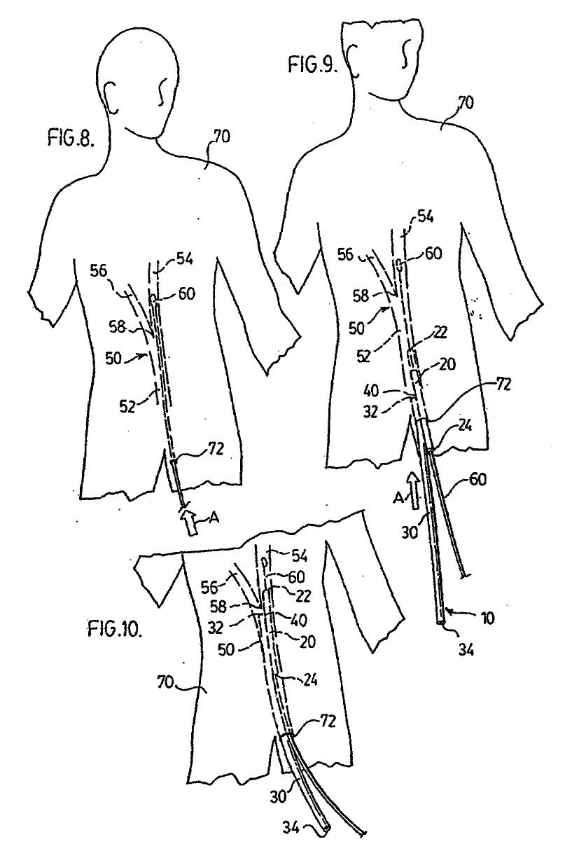

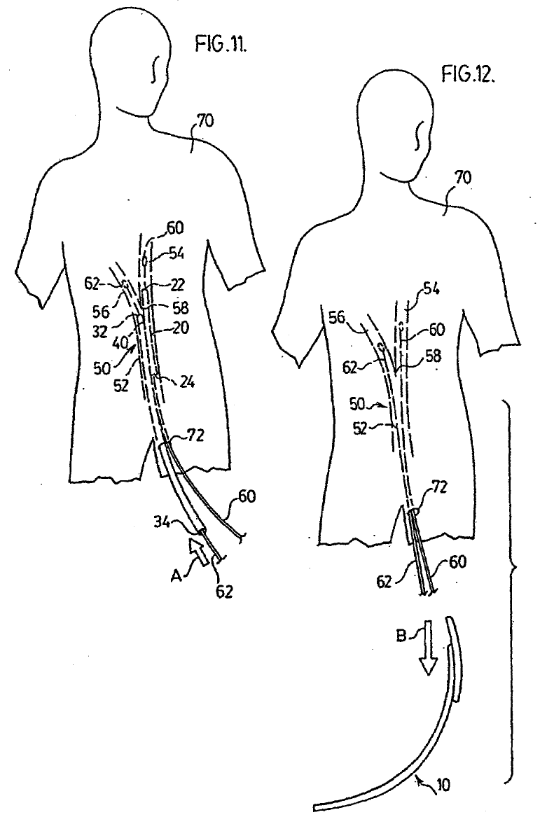

FIG. 8 concords with FIG. 3;
FIGS. 9 and 10 concord with FIG. 4;
FIG. 11 concords with FIG. 5; and
FIG. 12 concords with FIGS. 6 and 7.

As discussed above, endovascular sleeve 10 may be regarded as an “over-the-wire/monorail” delivery system. By this it is meant that, once the sleeve is in the correct position, one tubular passageway (30) remains over a guidewire (62) such that the proximal end thereof (34) emanates from the subject whereas the proximal end (24) of the other tubular passageway (20) does not emanate from the subject. In other words, the section of the other tubular passageway (20) between the bifurcated body passageway (50) and incision (72) in the subject (70) does not completely cover the other guidewire (60).

As discussed above, endovascular sleeve 100 may be regarded as a “double over-the-wire” delivery system. By this is meant that, once the sleeve is in the correct position, both tubular passage ways (120, 130) remain over their respective guidewires (60, 62) such that the proximal end (24) of each tubular passageway (120, 130) emanates from the subject. In other words, both guidewires (60, 62) are substantially completely covered by endovascular sleeve 100.

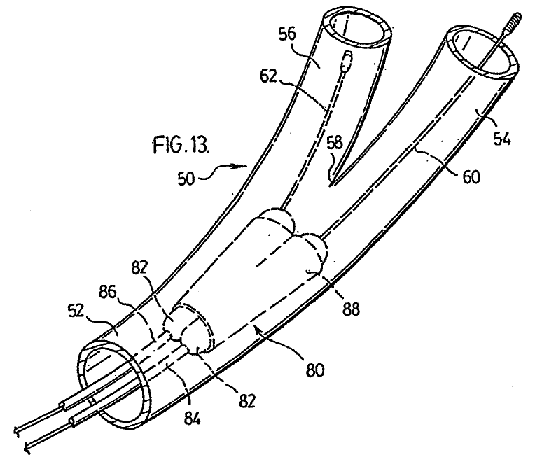

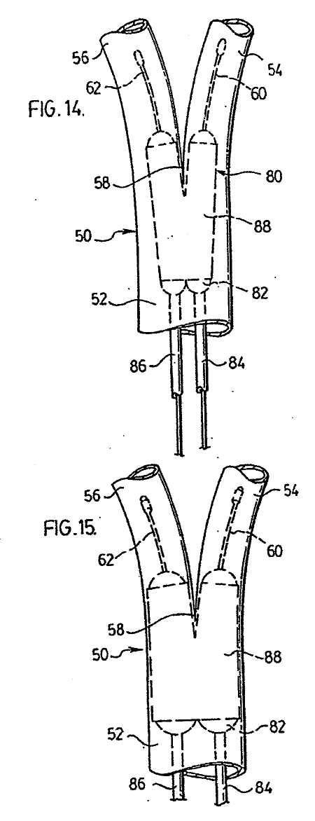

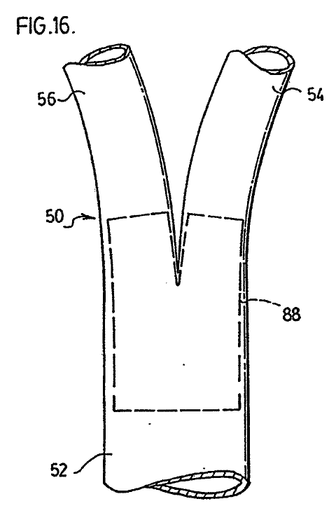

With reference to FIG. 7, once the endovascular sleeve is removed, guidewires 60, 62 remain as illustrated and are substantially untwisted to the point at which they emanate from the subject. With reference to FIG. 13, at this point, a catheter 80 is used to deliver a bifurcated stent to bifurcated body passageway 50. Specifically, catheter 80 comprises a balloon 82 having a pair of tubes 84, 86 emanating from one end thereof. Mounted on balloon 82 is a bifurcated stent 88. Tubes 84, 86 are of a conventional, annular design such that they can be disposed over their respective guidewires and can receive a fluid which is used to fill balloon 82 resulting in expansion thereof. Thus, catheter 80 is navigated over guidewires 60, 62 until the bifurcated stent is in the correct position—see FIG. 14. At this point, a pressurized fluid (e.g., saline) is introduced into balloon 82 via tubes 84, 86 resulting in expansion of balloon 82 and stent 88—see FIG. 15. Thereafter, balloon 82 is deflated conventionally and withdrawn from bifurcated body passage way 50 leaving stent 88 in a deployed state—see FIG. 16. While balloon 82 is shown as a pair of adjacent single balloons, those of skill in the art will appreciate that a bifurcated balloon could be used in place of a pair of single balloons.

While this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments.

What is claimed is:

1. An endovascular sleeve for delivering a pair of guidewires to a bifurcated body passageway of a patient, the sleeve comprising
a first tubular passageway and a second tubular passageway fixed with respect to one another, each tubular passageway configured to deliver a respective one of the pair of guidewires;
the first tubular passageway comprising a first distal end and a first proximal end, the second tubular passageway comprising a second distal end and a second proximal end;
the first distal end extending distally beyond the second distal end to define a junction which is configured to abut against a crotch in the bifurcated body passageway of the patient, the second distal end being fixed to said first tubular passageway at said junction; and
the second tubular passageway having a length such that the second proximal end extends from the patient.

2. The endovascular sleeve defined in claim 1, further comprising a radioopaque marker disposed thereon.

3. The endovascular sleeve defined in claim 2, wherein the radioopaque marker is disposed at the junction.

4. The endovascular sleeve defined in claim 1, wherein the first passageway has a substantially circular cross-section.

5. The endovascular sleeve defined in claim 1, wherein the second passageway has a substantially circular cross-section.

6. The endovascular sleeve defined in claim 1, wherein each of the first passageway and the second passageway has a substantially circular cross-section.

7. The endovascular sleeve defined in claim 1, wherein the first distal end extends beyond the second distal end by a margin of at least about 0.3 cm.

8. The endovascular sleeve defined in claim 1, wherein the fist distal end extends beyond the second distal end by a margin in the range of from about 0.3 to about 3 cm.

9. The endovascular sleeve defined in claim 1, wherein the first distal end extends beyond the second distal end by a margin in the range of from about 0.5 to about 2 cm.

10. The endovascular sleeve defined in claim 1, wherein the first distal end is chamfered.

11. The endovascular sleeve defined in claim 1, wherein the second distal end is chamfered.

12. The endovascular sleeve defined in claim 1, wherein each of the first distal end and the second distal end is chamfered.

13. A bifurcated stent delivery system for delivery of an expansible prosthesis to a bifurcated body passageway of a patient, the system comprising:
a catheter;
a pair of guidewires; and
an endovascular sleeve comprising a first tubular passageway and a second tubular passageway fixed with respect to one another, each tubular passageway configured to deliver a respective one of the pair of guidewires,
the first tubular passageway comprising a first distal end and a first proximal end, the second tubular passageway comprising a second distal end and a second proximal end,
the first distal end extending distally beyond the second distal end to define a junction which is configured to abut against a crotch in the bifurcated body passageway of the patient, the second distal end being fixed to said first tubular passageway at said junction,
the second tubular passageway having a length such that the second proximal end extends from the patient.

14. The system defined in claim 13, wherein the endovascular sleeve further comprises a radioopaque marker disposed thereon.

15. The system defined in claim 14, wherein the radioopaque marker is disposed at the junction.

16. The system defined in claim 13, wherein the first passageway has a substantially circular cross-section.

17. The system defined in claim 13, wherein the second passageway has a substantially circular cross-section.

18. The system defined in claim 13, wherein each of the first passageway and the second passageway has a substantially circular cross-section.

19. The system defined in claim 13, wherein the first distal end is at least about 0.3 cm is longer than the second distal end.

20. The system defined in claim 13, wherein the first distal end is longer than the second distal end by a margin in the range of from about 0.3 to about 3 cm.

21. The system defined in claim 13, wherein the first distal end is longer than the second distal end by a margin in the range of from about 0.5 to about 2 cm.

22. The system defined in claim 13, wherein the first distal end is chamfered.

23. The system defined in claim 13, wherein the second distal end is chamfered.

24. The system defined in claim 13, wherein each of the first distal end and the second distal end is chamfered.

25. The system defined in claim 13, wherein the catheter comprises at least one expandable member.

26. The system defined in claim 25, wherein the expandable member is disposed adjacent a distal end of the catheter.

27. The system defined in claim 25, wherein the catheter comprises two expandable members.

28. The system defined in claim 25, wherein the catheter comprises a substantially Y-shaped expandable member.

29. The system defined in claim 25, wherein the expandable member comprises a balloon.

30. The system defined in claim 25, further comprising a bifurcated stent disposed on the expandable member.

31. The system defined in claim 30, wherein the bifurcated stent is mounted on the expandable member.

32. The endovascular sleeve defined in claim 1, wherein the second proximal end extends beyond the first proximal end.

33. The endovascular sleeve defined in claim 32, wherein the first tubular passageway has a length such that the first proximal end does not emanate from a subject and the second tubular passageway has a length such that the second proximal emanates from the subject.

34. The endovascular sleeve defined in claim 1, wherein first proximal end and the second proximal end are substantially juxtaposed.

35. The endovascular sleeve defined in claim 34, wherein the first tubular passageway and the second tubular passageway each have a length such that the first proximal end and the second proximal end each emanate from a subject.

36. The endovascular sleeve defined in claim 1, wherein the first tubular passageway and the second tubular passageway are each constructed of a material having sufficient integrity to be navigated through tortuous body passageways.
